(19) United States
(12) Patent Application Publication
(10) Pub. No.: US 2007/0031412 A1
(43) Pub. Date: Feb. 8, 2007

(54) Direct and indirect effector cell protease receptor-1 (EPR-1) Inhibitors as antiplatelet agents

(76) Inventors: Javier Pedreno Egea, Barcelona (ES); Luis Caveda Catasus, Miami, FL (US)

Correspondence Address: MOORE & VAN ALLEN PLLC, P.O. BOX 13706, Research Triangle Park, NC, 27709, US

(21) Appl. No.: 11/498,347

(22) Filed: Aug. 3, 2006

Related U.S. Application Data
(60) Provisional application No. 60/719,643, filed on Sep. 21, 2005.

(30) Foreign Application Priority Data
Aug. 3, 2005 (EP) 05380179.1

(51) Int. Cl. A61K 39/395; A61K 38/37

(52) U.S. Cl. 424/145.1; 514/12

(57) ABSTRACT

Direct and indirect effector cell protease receptor-1 (EPR-1) inhibitors may be used for the treatment of conditions associated with platelet aggregation as antiplatelet agents.

### CROSS REFERENCE TO RELATED APPLICATIONS

This application claims priority under 35 U.S.C. §119(a) of European Patent Application No. 05380179.1 for “Activated Factor X Stimulants as New Antihemorrhagic Agents for Topical Use,” filed on Aug. 3, 2005 in the name of Javier Pedreño Egea et al., and under 35 U.S.C. §119(e) of U.S. Provisional Patent Application No. 60/719,643 for “Activated Factor X Stimulants as New Antihemorrhagic Agents for Topical Use,” filed on Sep. 21, 2005 in the name of Javier Pedreño Egea et al., both of which are incorporated by reference herein in their entirety.

### FIELD OF THE INVENTION

This invention relates to the use of molecules which directly or indirectly inhibit or block the activity of the effector cell protease receptor-1 (EPR-1) for the treatment of conditions associated with platelet activation and/or platelet aggregation as antiplatelet agents.

### BACKGROUND OF THE INVENTION

1. Physiology of Coagulation

Hemostasis is the mechanism by means of which living beings respond to a hemorrhage and involves the participation of two processes that become functional immediately after a lesion and remain active for a long period of time. The first of them is known as primary hemostasis and is characterized by the occurrence of vasoconstriction at the vascular lesion site and platelet aggregate formation. The second one is known as secondary hemostasis, being the phase in which the fibrin clot is formed due to the action of the different coagulation cascade proteolytic enzymes.

Platelet aggregate formation plays a key role in hemostasis in capillaries, being particularly relevant in mucocutaneous hemorrhaging; in contrast, fibrin clot formation is much more important in large vessel hemostasis, being more relevant in internal hemorrhaging (gastrointestinal, cerebral, etc.). The following phases can be distinguished during platelet aggregate formation: (i) platelet adhesion to the sub-endothelium surface exposed by the lesion; (ii) release of the granular content of platelets as a response to their activation; (iii) platelet aggregation with the subsequent sequestering and concentration of more platelets at the lesion site; and (iv) binding of fibrinogen as well as other coagulation proteins to the platelet surface to produce thrombin and form the fibrin clot that will allow the plates to become fused and consolidated, thus stabilizing the hemostasic clot. It is well known that platelet count is critical for fibrin clot formation; counts below 20,000 per μl are accompanied with severe bleeding episodes.

Several cofactors and proteolytic enzymes participate in the second phase of the blood coagulation process, all referred to as coagulation factors, and it consists of several phases ending with fibrin formation from fibrinogen hydrolysis due to the action of thrombin. Furthermore, the thrombin production enhances the platelet aggregate by increasing the activation and aggregation of more platelets. Thrombin is previously formed by proteolytic hydrolysis of an apoenzyme, prothrombin. This proteolysis is carried out by factor X activated (FXa), which binds to the surface of the activated platelets and only in the presence of its cofactor, activated coagulation factor V (FVa), and calcium ions, is able to hydrolyze prothrombin and to start blood coagulation. Moreover, it is well known that FXa when is bound to the membrane surface may cause directly platelet aggregation but the underlying mechanism remain understood. However, one may think that FXa indirectly enhances platelet aggregation through proteolytic hydrolysis of prothrombin and consequent formation of thrombin. Therefore, FXa by both direct and indirect mechanism may act as a strong activator of platelet aggregation.

FXa can occur in two separate pathways, the intrinsic pathway and the extrinsic pathway. Intrinsic pathway consists of a series of reactions involving mainly coagulation factor VIII (FVIII), coagulation factor IX (FIX) and coagulation factor XI (FXI), in which each proenzyme is hydrolyzed, yielding its active protease form (FVIIIa, FIXa and FXIa). In each step, the recently formed proteolytic enzyme will catalyze the activation of the following proenzyme to successively yield the active form. Activation of the different coagulation factors involved in the intrinsic pathway takes place, and overall leads to activation of FX.

Extrinsic pathway is activated when the TF exposed on adventitia cells at the lesion site, binds to circulating coagulation factor VII/activated coagulation factor VII (FVII/FVIIa) to form the TF::FVIIa complex and, in the presence of calcium, to act as a substrate so that FX activation takes place. TF is an integral membrane glycoprotein belonging to the super-family of class II cytokine receptors specifically bind FVII/FVIIa and plays a relevant role in the blood coagulation extrinsic pathway. The physiological roles assigned to TF are well known; on the one hand, it is a receptor specific for FVIIa and, once the TF::FVIIa complex has been formed, it acts as a substrate so that FX activation takes place. In fact, after a vascular lesion, TF comes into contact and interacts with its ligand, FVII, present in blood, to form the TF::FVII complex. Once this complex is formed, FVII autoactivation takes place, yielding its active form FVIIa. The extrinsic pathway is currently considered the most relevant pathway in blood coagulation, and it is accepted that in the event of a hemorrhage produced by a vascular lesion, coagulation is triggered due to extrinsic pathway activation involving the interaction of TF with its ligand, FVII/FVIIa. Another role assigned to the TF::FVIIa complex in coagulation is to act as a substrate so that FX activation takes place due to FVIIa. As a result, basal FXa levels (<150 pM), which initially are insufficient to generate fibrin clot formation, increase. This increase in basal FXa concentrations in the presence of its cofactor, FVa, and of a cellular procoagulant surface, would be able to produce the thrombin required for fibrin clot formation. It is currently accepted that once the platelets are activated provide the procoagulant surface rich in anionic phospholipids and they expose the FVa and FXa factors stored within them. All this allows correct assembly of the different agents involved in coagulation on the surface of their plasma membranes forming the well known prothrombinase complex (FXa, FVa, prothrombin, and anionic procoagulant platelet phospholipids surface).

Therefore, FXa plays a key role in thrombosis and hemostasis by enzymatically cleaving its substrate, prothrombin, to produce thrombin, thereby regulating the generation of this vital procoagulant enzyme. Despite the vital role of FXa in thrombosis is rather well defined; its contribution to other physiological and pathophysiological mechanisms is only just beginning to unfold. Increasing evidence indicates that FXa elicits numerous and profound cellular events, such as release of cytokines, expression of adhesion molecules, tissue factor gene expression and cell proliferation. These cellular responses are mediated by specific receptors and involve various intracellular signaling molecules and/or extracellular mediators. Thus, the binding of FXa to cells and the subsequent cellular responses may contribute to diseases such as arterial restesnosis, acute inflammation, sepsis and cancer. FXa-induced cellular signaling may be mediated, in part, by a cellular binding site for FXa, referred to as effector cell protease receptor-1 (EPR-1). FXa binding to EPR-1 expressed by cell populations occurs independently of factor Va. EPR-1 is a 65-kDa membrane-spanning receptor originally identified on monocytes and various leukocytes as a novel cell-surface receptor for FXa. EPR-1 was later shown to be expressed on vascular endothelial cells and smooth muscle cells. Recently it has been reported that EPR-1 is expressed on platelet surface regulating thrombin formation, thus regulating coagulation. There are different theories about the mechanism through which EPR-1 regulates thrombin generation, the most plausible is that EPR-1 mediates FXa assembly into the prothrombinase complex and is required with FVa to mediate FXa binding to the activated platelet form a functional prothrombinase complex.

2. Platelet Activation and Early-Phases of Platelet Aggregation

Platelets play a central role in the process of thrombus formation, as well as an important role in atherogenesis and the progression of atherosclerotic lesions.

The interaction of the platelet with the vessel wall and its subsequent contribution to atheroma formation and thrombosis is of pivotal importance in the etiology and pathogenesis of peripheral, coronary, cerebrovascular and other vascular diseases. Acknowledgement of the fact that platelets have a central role to play in these disease states has led to a considerable amount of research into its pathophysiology. Indeed, inappropriate platelet activation is common in atherosclerosis, and many of its risk factors, such as smoking and diabetes. Platelet activation comprises a change in platelet shape, early phases of platelet aggregation and the release of platelet constituents. Once a platelet agonist, for example thrombin, has bound its receptor on platelet surface, the signal transduction initiates the activation process and platelet release reaction and subsequently platelet activation takes place. The role of antiplatelet therapy in reducing the risk of many cardiovascular and cerebrovascular disorders is also well established. So, it may conclude that inappropriate platelet activation plays a prime role in the increasing heart disease burden of society. Platelet activation has been implicated in the pathogenesis of a number of diseases, which includes atherosclerosis, coronary vascular disease and cerebrovascular disease. Abnormal platelet activation has also been associated with atrial fibrillation, cancer, peripheral vascular disease, Alzheimer disease, inflammatory bowel disorders and recently deep vein thrombosis. In conclusion, a lot of systemic diseases occurring with alterations in platelet activation and early-phases of platelet aggregation have been disclosed.

There has been a great deal of progress in the understanding of molecular and biological mechanisms involved in platelet activation, that is, platelet vessel wall interaction, platelet-platelet interaction, and subsequent clot formation. This has led to the localization and identification of various targets for interruption of platelet activation. A greater understanding of platelet activation has also helped the development of a number of antiplatelet drugs. By assessing the effect on an antiplatelet mediation on platelet activation, it may be possible to choose the appropriate antiplatelet therapy for an individual and subsequently assess the impact of introduction of the medication or to estimate the appropriate dose.

Due to the pivotal role of platelets in thrombus formation, especially in the arterial system (thrombus affecting arterials is platelet rich thrombus whereas thrombus in venous vessels is hyaline), inhibition of platelet activation and early-phases of platelet aggregation has become a central pharmacological approach. This is done to prevent and treat thromboembolic diseases such as coronary heart disease, peripheral and cerebrovascular disease and is also used during as well as after invasive coronary interventions.

The effectiveness of the present antiplatelet drugs is variable, this remaining a clinical unmet need. ADP receptor antagonists and glycoprotein IIb/IIIa antagonists do not prevent atherosclerosis and restenosis after angioplasty or stent implant. Aspirin and clopidrogel reduce thrombotic occlusion in unstable plaque leading to stroke and myocardial infarctions, but many patients are still admitted with unstable coronary and carotid syndromes with associated progression to myocardial infarction, cerebrovascular accident or peripheral vascular occlusion, what suggests that their activity is only partially effective in preventing disease progression.

Therefore there is an unmet need for a simultaneously safe and effective clinical agent as antiplatelet agent that can act, if desired, as combined antithrombotic and early-stage antiplatelet agent.

3. Background of the Invention

The vital role of FXa in thrombosis is rather well defined; however, its contribution to other physiological and pathophysiological mechanisms is only just beginning to unfold. Increasing evidence indicates that FXa elicits cellular events mediated by specific receptors referred to as EPR-1 Thus, the binding of FXa to EPR-1 and the subsequent cellular responses may contribute to diseases such as arterial restenosis, acute inflammation, sepsis and cancer. EPR-1 has been identified on monocytes, leukocytes, vascular endothelial cells and smooth muscle cells. Recently it has been reported that EPR-1 is also expressed on platelet surface mediating FXa binding to the activated platelet and to form the functional prothrombinase complex.

The inventors have now demonstrated for the first time that platelet EPR-1 directly regulates platelet aggregation through specific binding of FXa (stored and released by activated platelets or exogenously added) and surprisingly, in this process TF is involved.

Therefore, the inventor's discovery opens the door for the treatment of platelet activation and platelet aggregation mediated diseases with direct EPR-1 inhibitors or antagonists (e.g., antibodies against EPR-1) or indirect EPR-1 inhibitors or antagonists (e.g., antibodies against FXa and TF).

WO 03/093422 discloses an anticoagulant antibody against TF that binds with greater affinity to FVIIa/TF complex than to TF alone, and that inhibits thrombin generation without directly affecting other coagulation parameters such as the activation and aggregation of platelets. WO 03/070275 discloses the use of an anti-TF antibody for the treatment of diabetes. WO 03/037911 and WO9640921 disclose anti-TF antibodies for their use as anticoagulants. Nevertheless, the use of said anti-TF antibodies as antiplatelet agents has not been described in said patents.

U.S. Pat. No. 6,274,142 discloses methods and therapeutic compositions for the treatment of myocardial infarction by means of an anticoagulant and a thrombolitic agent, being said anticoagulant a tissue factor protein antagonist (such as an antibody). U.S. Pat. No. 5,679,639 discloses the use of anti-FXa antibodies as anticoagulant agents. U.S. Pat. No. 6,238,875 discloses anti-EPR-1 antibodies and their use as diagnostic methods. None of these antibodies has been described as antiplatelet agents.

The TF antibody described in the present patent application has high affinity to TF and specifically affects platelet activation and aggregation in addition to its anticoagulant activity. The new antiplatelet agents hereby disclosed and claimed can act as early stage antiplatelet agents preventing platelet activation and inhibiting platelet aggregate clot formation.

### SUMMARY OF THE INVENTION

Despite the vital role of FXa in thrombosis is rather well defined; its contribution to other physiological and pathophysiological mechanisms remains to be investigated. Increasing evidence indicates that FXa is participating in relevant cellular responses mediated by its specific membrane receptor, EPR-1. Thus, the binding of FXa to cells may contribute to diseases such as arterial restesnosis, acute inflammation, sepsis and cancer. However, until now, nobody has still postulated EPR-1 as a modulator of platelet activation and aggregation.

In the present patent application, the inventors have demonstrated for the first time that platelet EPR-1 directly regulates platelet activation and aggregation through specific binding of FXa (stored and released by activated platelets or exogenously added). Therefore, this finding opens the door for the treatment of platelet activation and platelet aggregation mediated diseases with direct EPR-1 inhibitors (e.g., molecules with anti-EPR-1 activity) or with indirect EPR-1 inhibitors (e.g., molecules with anti-FXa activity).

On the other hand, the inventors have demonstrated that lipidated TF alone is able to induce a strong platelet aggregation effect in activated platelets (which express p-selectin). This aggregatory effect is fully blocked by molecules with anti-TF activity. Surprisingly, monoclonal antibodies against FXa and polyclonal antibodies against EPR-1 were able to block completely the aggregatory effect mediated by TF. These results clearly indicate that lipidated TF is able to aggregate platelets by means of a mechanism involving the participation of intraplatelet FXa (or exogenous added) and its membrane platelet receptor, EPR-1. Moreover, TF is able to cause a significant increase in the platelet FXa proaggregatory effect. Low FXa concentrations unable to produce aggregation by itself (<10 pM) in the presence of lipidated TF (50 ng of active protein/ml) are able to cause a strong platelet aggregation effect. The inventors have discovered that despite to the accepted role of TF as membrane receptor for FVII, TF is also a potent FXa stimulator even in the absence of FVII/FVIIa. TF acts as stimulator of FXa producing a significant enhance in its proteolytic and platelet proaggregatory activities. Thus, the inventor's discovery opens the door for the treatment of platelet activation and platelet aggregation mediated diseases with other indirect EPR-1 inhibitors, e.g., molecules with anti-TF activity.

Therefore, compounds having anti-FXa activity, anti-TF activity or anti-EPR-1 activity, such as, for example, anti-FXa antibodies, anti-TF antibodies and anti-EPR-1 antibodies, are effective as antiplatelet agents in the treatment of platelet associated thromboembolic diseases, especially those wherein platelet activation plays a relevant role such as atherosclerosis, coronary vascular and cerebrovascular diseases, atrial fibrillation, cancer, peripheral vascular disease, Alzheimer disease, inflammatory bowel disorders and deep vein thrombosis.

This capital finding opens the door to a new family of platelet regulators based on the direct or indirect inhibition of EPR-1.

At present, many of known antiplatelet agents are thrombin-induced antiplatelet agents, thus meaning that significant increase of thrombin concentrations must be achieved to reach the antiplatelet properties of said agents. Consequently, the antiplatelet action begins once the platelets are activated and aggregated, and the blood clot is already formed.

Therefore, in an aspect, the invention relates to the use of a new family of antiplatelet agents, named as EPR-1 inhibitors or antagonists. The inventors have classified EPR-1 inhibitors or antagonists in two classes: (i) direct inhibitors or antagonists, and (ii) indirect inhibitors or antagonists. If the inhibition of cellular responses mediated by EPR-1 is achieved by molecules directed to EPR-1, said molecules are classified as “direct EPR-1 inhibitors or antagonists”. However, if the inhibition of cellular responses mediated by EPR-1 is achieved by molecules (e.g., antibodies) directed to a ligand of EPR-1 (e.g., FXa) or directed to a stimulator of a ligand of EPR-1 (e.g., TF), said molecules are classified as “indirect EPR-1 inhibitors or antagonists”.

Thus, in an aspect, the invention relates to the use of an EPR-1 effector in the manufacture of an antiplatelet pharmaceutical composition. In an embodiment, said antiplatelet pharmaceutical composition is used for the prevention and/or treatment of a condition associated with platelet aggregation in a subject. In another embodiment, said EPR-1 effector is a direct or indirect EPR-1 inhibitor or antagonist. Further, in an specific embodiment, said EPR-1 inhibitor or antagonist is an antibody against EPR-1 or an antigen-binding fragment thereof, or an antibody against FXa or an antigen-binding fragment thereof, or an antibody against TF or an antigen-binding fragment thereof.

In other aspect, the invention relates to a method for forming a non-thrombogenic coating in the surface of a medical device for surgical operations which comprises contacting the surface of said medical device with an EPR-1 inhibitor or antagonist.

In another aspect, the invention relates to a method for treating a condition associated with platelet activation and/or platelet aggregation, or a thrombus or embolus mediated disease, which comprises administering to a subject in need of said treatment a therapeutically effective amount of an EPR-1 effector, such as an EPR-1 inhibitor or antagonist.

### DETAILED DESCRIPTION OF THE INVENTION

The interaction of the platelet with the vessel wall and its subsequent contribution to atheroma formation and thrombosis is of pivotal importance in the etiology and pathogenesis of peripheral, coronary, cerebrovascular and other vascular diseases. Once a platelet agonist has bound to its receptor on the platelet surface, the signal transduction initiates the platelet release reaction and subsequently platelet activation takes place. Platelet activation comprises a change in platelet shape, early phases of platelet aggregation and the release of platelet constituents. Inappropriate platelet activation has been implicated in the pathogenesis of a number of diseases, which includes atherosclerosis, coronary vascular disease and cerebrovascular disease, playing a prime role in the increasing heart disease burden of society. Moreover, abnormal platelet activation has also been associated with atrial fibrillation, cancer, peripheral vascular disease, Alzheimer disease, inflammatory bowel disorders and recently with deep vein thrombosis. In conclusion, a lot of systemic diseases occurring with alterations in platelet activation and early-phases of platelet aggregation have been disclosed.

Current anticoagulant therapies focus on the latter stages of the clotting cascade. All of the known antiplatelets drugs decrease the risk of cardiovascular and cerebrovascular disorders reducing platelet aggregation, but only a few number of antiplatelet drugs have demonstrated efficiency to inhibit both platelet activation and early-phase of platelet aggregation. At the present, there is a substantial demand for the development of novel therapeutics that target earlier events on all these pathways (coagulation, platelet activation and platelet aggregation). Therefore, it is an object of this invention to provide compositions and methods for antiplatelet therapy focused in early-phases of platelet aggregation and activation.

As mentioned above, the present invention is based on the finding that EPR-1 directly regulates platelet aggregation by interaction with its specific ligand, FXa (either stored and released by activated platelets or exogenously added) and, surprisingly, TF is also involved in this process.

The inventors have now shown that FXa at picomolar concentrations below 10 pM are unable to produce significant effects in coagulation, platelet activation and platelet aggregation, even in the presence of its well known cofactor, FVa. On the other hand, it has also been shown that FXa at concentrations around 10-150 pM are able to induce only platelet aggregation, and thrombin formation takes place only in the presence of higher concentrations (>1,500 pM). Therefore, concentrations of FXa between 10 pM and 1,500 pM (interval containing the postulated basal FXa concentrations of 150 pM) are able to induce platelet aggregation without affecting thrombin formation. However, in the presence of TF, FXa even at low concentrations (below 10 pM), is able to alter platelet function inducing a strong platelet aggregation effect. The aggregatory effect induced by FXa is 50-fold more potent than thrombin. Moreover, a synergistic effect between both proteolytic enzymes was observed at concentrations unable to produce aggregation by itself. These results indicate that probably the expression of intraplatelet FXa (stored in α-granules) and the binding to EPR-1 on surface of activated platelets plays a key role in the early phase of platelet activation process. After, EPR-1 initiates the transduction signal enhancing platelet release reaction and aggregation. The inventors have demonstrated that the FXa-induced aggregatory effect is fully inhibited by monoclonal antibodies against FXa or polyclonal antibodies against EPR-1 (Example).

Therefore, the inventor's discovery opens the door for the treatment of platelet activation and/or platelet aggregation mediated diseases with direct EPR-1 inhibitors (e.g., molecules having anti-EPR-1 activity) or antagonists or with indirect EPR-1 inhibitors or antagonists (e.g., molecules having anti-FXa activity).

On the other hand, the inventors have also demonstrated that despite to the accepted role of TF as membrane receptor for FVII, this protein acts as a potent FXa stimulator even in the absence of FVII/FVIIa. This stimulator effect produces that FXa increases significantly its proteolytic activity and platelet aggregatory effects.

Until now no role in platelet aggregation for TF alone had been assigned. The inventors have demonstrated that lipidated TF alone is able to induce a strong platelet aggregation effect in activated platelet (expressing p-selectin). This aggregatory effect is fully blocked by anti-TF antibodies. Surprisingly, monoclonal antibodies against FXa or polyclonal antibodies against EPR-1 were able to block the proaggregatory effect mediated by TF. These results clearly indicates that lipidated TF is able to aggregate platelets by means of a mechanism involving the participation of intraplatelet FXa (or exogenous) and its membrane platelet receptor, EPR-1. Moreover, TF is able to cause a significant increase in the platelet FXa proaggregatory effect. Low FXa concentrations unable to produce aggregation by itself (<10 pM) in the presence of lipidated TF (50 ng of active protein/ml) cause a strong platelet aggregation effect.

The inventor's discovery opens the door for the treatment of platelet activation and platelet aggregation mediated diseases with other indirect EPR-1 inhibitors, e.g., molecules with anti-TF activity.

Therefore, the inventors have demonstrated the inhibition of the proaggregatory effects mediated by EPR-1 by means of: (1) polyclonal antibodies against EPR-1, (2) monoclonal antibody against its natural ligand, FXa, and (3) monoclonal antibodies against TF. These antibodies are effective as antiplatelet agents for the prevention and treatment of thrombus formation; thus, said antibodies could be used for preventing and/or treating conditions associated with platelet activation and/or platelet aggregation in a subject.

The inventors have now demonstrated the inhibition of the proaggregatory effects mediated by EPR-1 by means of antibodies against EPR-1, antibodies against FXa, and antibodies against TF. Therefore, all these antibodies (anti EPR-1, anti FXa and anti TF) are effective as antiplatelet agents in the prevention and/or treatment of conditions associated with platelet activation and/or platelet aggregation in a subject, such as platelet associated thromboembolic diseases, especially those in which platelet activation plays a relevant role such as atherosclerosis, coronary vascular and cerebrovascular diseases, atrial fibrillation, cancer, peripheral vascular disease, Alzheimer disease, inflammatory bowel disorders and deep vein thrombosis.

This capital finding opens the door to a new family of platelet regulators based on the direct or indirect inhibition of the activity of EPR-1.

Consequently, in an aspect, the invention relates to the use of an EPR-1 effector in the manufacture of an antiplatelet pharmaceutical composition; said antiplatelet pharmaceutical composition can be used for the prevention and/or treatment of a condition associated with platelet aggregation in a subject.

The term “subject”, as used herein, includes any member of an animal species, including the human species; by way of an illustrative, non-limiting example, said subject can be a mammal, such as a primate, a domestic animal, a rodent, etc., said subject is preferably a man or woman of any age and race. In a particular embodiment said subject is a human being with no history of hemostasis disorders, such as an individual having no coagulopathies or platelet disorders. In another particular embodiment, said subject is a human being having a history of hemostasis disorders, such as an individual having hemorrhagic diathesis, for example, a platelet disorder, such as a congenital or acquired platelet disorder. In another particular embodiment, said subject is a human being having a condition associated with platelet activation and/or platelet aggregation. In another particular embodiment, said subject is a human being having a platelet aggregation mediated disease, such as thrombus or embolus mediated diseases.

The term “condition associated with platelet activation and/or platelet aggregation”, as used herein, includes, among others, any disorder or condition related to an inappropriate or abnormal platelet activation and/or platelet aggregation. Illustrative, non-limitative examples of conditions or disorders associated with an inappropriate platelet activation include atherosclerosis, coronary vascular disease and cerebrovascular disease. Illustrative, non-limitative examples of conditions or disorders associated with an abnormal platelet activation include atrial fibrillation, cancer, peripheral vascular disease, Alzheimer disease, inflammatory bowel disorders and recently with deep vein thrombosis.

The term “thrombus or embolus mediated diseases”, as used herein, refers to a disorder or condition due to the formation of a thrombus inside a vessel, and/or to the detachment of a part of the clot (embolus) this flowing to smaller vessels that can be completely obtruded avoiding blood circulation, producing an infarction of the part of the body that suffers this lack of blood supply. In a particular embodiment, said thrombus or embolus mediated disease is a thromboembolic disease such as coronary heart disease, acute deep venous thrombosis, pulmonary embolism, acute arterial embolization of the extremities, myocardial infarction, disseminated intravascular coagulation, coronary artery disease, peripheral and cerebrovascular disease, intermittent claudication, thromboembolic complications of surgery (during as well as after invasive coronary interventions), or recurrence of embolism in patients with rheumatic and arteriosclerotic heart disease.

As used herein, the term “EPR-1 effector” refers to any molecule that has or exerts some effect on EPR-1 activity. Said EPR-1 effector can be a direct EPR-1 effector, i.e., a molecule that has or exerts some effect directly on the EPR-1 activity, or an indirect EPR-1 effector, i.e., a molecule that has or exerts some effect on the EPR-1 activity through its interaction with a ligand of EPR-1, e.g., FXa (i.e., said molecule is herein considered, in general, as a FXa effector) or through its interaction with a stimulator of a ligand of EPR-1, e.g., TF (i.e., said molecule is herein considered, in general, as a TF effector).

In a particular embodiment, said EPR-1 effector is a direct or indirect EPR-1 inhibitor or antagonist, i.e., a molecule which inhibits or blocks the EPR-1 activity. Said inhibition or blocking may be total or partial. Virtually any molecule which inhibits or blocks the EPR-1 activity, either directly, i.e., interacting directly with EPR-1, or indirectly by interacting with an EPR-1 ligand, e.g., FXa, or interacting with a stimulator of an EPR-1 ligand, e.g., TF, can be used within the context of the instant invention. Thus, in a particular embodiment, said EPR-1 inhibitor or antagonist can be, for example, a molecule which binds to EPR-1, or to a ligand thereof (e.g., FXa) or to a stimulator of an EPR-1 ligand (e.g., TF) and inhibits or blocks the EPR-1 activity. Illustrative, non-limitative examples of molecules which can be used as EPR-1 inhibitors or antagonists include small organic compounds, peptides, fusion proteins, antibodies or antigen-binding fragments thereof, etc.

As used herein, the term “antibody”, refers to an immunoglobulin, or a fragment thereof, that exhibits a specific binding activity for a target molecule, namely, EPR-1, a ligand thereof (e.g., FXa) or a stimulator of an EPR-1 ligand (e.g., TF), hereinafter referred to as the “target molecule”. Thus, said antibody is capable of binding an epitope of the target molecule [typically, at least 6, 8, 10, or 12, contiguous amino acids are required to form an epitope, however, epitopes which involve non-contiguous amino acids may require more, e.g., at least 15, 25, or 50 amino acid]. The term “antibody” includes, for example, monoclonal antibodies, polyclonal antibodies, engineered or modified antibodies, chimeric antibodies, humanized antibodies, primatized antibodies, human antibodies, antibody fragments (e.g., Fab, F(ab′)₂, Fab′, scFv, etc.), diabodies, bispecific antibodies (including cross-linked or heteroconjugate antibodies), etc.

Such antibodies may be produced in a variety of ways, including hybridoma cultures, recombinant expression in bacteria or mammalian cell cultures, and recombinant expression in transgenic animals. Also antibodies can be produced by selecting a sequence from a library of sequences expressed in display systems such as filamentous phage, bacterial, yeast or ribosome. Fragments of antibodies can be produced, for example, by proteolytic digestion. There is abundant guidance in the literature for selecting a particular production methodology, e.g., Chadd and Chamow, Curr. Opin. Biotechnol., 12:188-194 (2001).

Thus, in a particular embodiment, the EPR-1 inhibitor or antagonist is an antibody, or an antigen-binding fragment thereof, that exhibits a specific binding activity for EPR-1.

In other particular embodiment, the EPR-1 inhibitor or antagonist is an antibody, or an antigen-binding fragment thereof, that exhibits a specific binding activity for an EPR-1 ligand, e.g., for FXa, i.e., said EPR-1 inhibitor or antagonist is an antibody, or an antigen-binding fragment thereof, that exhibits a specific binding activity for FXa.

In another particular embodiment, the EPR-1 inhibitor or antagonist is an antibody, or an antigen-binding fragment thereof, that exhibits a specific binding activity for a stimulator of an EPR-1 ligand, e.g., for TF, i.e., said EPR-1 inhibitor or antagonist is an antibody, or an antigen-binding fragment thereof, that exhibits a specific binding activity for TF.

In a particular embodiment, said antibody is a monoclonal antibody.

As mentioned above, said EPR-1 inhibitor or antagonist can be used as an antiplatelet agent, for example, in an embodiment, said EPR-1 inhibitor or antagonist can be used for the prevention and/or treatment of a condition associated with platelet activation and/or platelet aggregation in a subject, for example, by inhibiting the platelet activation in said subject or by inhibiting the platelet aggregation in said subject, or for inhibiting thrombus or embolus formation in a subject.

In other particular embodiment, said EPR-1 inhibitor or antagonist can be used for the prevention and/or treatment of a cardiovascular disease related to thrombosis or embolism in a subject, or for the prevention and/or treatment of a cardiovascular abnormal condition related to thrombosis or embolism in a subject.

In other particular embodiment, said EPR-1 inhibitor or antagonist can be used for the prevention and/or treatment of a platelet associated thromboembolic disease that is primary arterial in origin in a subject, for example, for the prevention and/or treatment of recurrence myocardial infarctions and stroke in a subject or for the prevention and/or treatment of recurrence of embolism in patients with rheumatic and/or arteriosclerotic heart disease.

In another particular embodiment, said EPR-1 inhibitor or antagonist can be used for the prevention and/or treatment of acute deep venous thrombosis, pulmonary embolism, acute arterial embolization of the extremities, myocardial infarction, disseminated intravascular coagulation, coronary artery disease and cerebrovascular disease in a subject.

In another particular embodiment, said EPR-1 inhibitor or antagonist can be used for the prevention and/or treatment of thromboembolic complications of surgery, for example, wherein said surgery includes hip replacement, angioplasty (e.g., coronary, pulmonary, peripheral, intracranial, extracranial carotid, renal, or aortic angioplasty) or invasive cardiovascular surgery, in particular, wherein said invasive cardiovascular surgery includes coronary artery bypass graft or heart valve replacement. Further, in another particular embodiment, said EPR-1 inhibitor or antagonist can be used to form a non-thrombogenic coating in the surface of a medical device used during said surgical operation.

In another particular embodiment, said EPR-1 inhibitor or antagonist can be used for the prevention and/or treatment of cancer (in general), optionally, in combination with an antineoplasic agent and a pharmaceutically acceptable carrier.

In another particular embodiment, said EPR-1 inhibitor or antagonist can be used for the prevention and/or treatment of Alzheimer disease (AD), optionally, in combination with an additional drug (e.g., a drug for treating AD or some symptoms thereof) and a pharmaceutically acceptable carrier.

In another particular embodiment, said EPR-1 inhibitor or antagonist can be also used for the prevention and/or treatment of atrial fibrillation, optionally, in combination with an additional drug (e.g., a drug for treating atrial fibrillation) and a pharmaceutically acceptable carrier.

For the administration of the EPR-1 effector to a subject, said compound should be formulated in a pharmaceutical composition. Thus, the invention further relates to a pharmaceutical composition comprising an EPR-1 effector together with a pharmaceutically acceptable carrier. In a particular embodiment, said EPR-1 effector is an EPR-1 inhibitor or antagonist, e.g., an antibody or an antigen-binding fragment thereof, that exhibits a specific binding activity for EPR-1, or for an EPR-1 ligand (e.g., FXa) or for a stimulator of an EPR-1 ligand (e.g., TF). Thus, in a particular embodiment, said antibody is selected from the group consisting of (i) an antibody against EPR-1 or an antigen-binding fragment thereof, (ii) an antibody against FXa or an antigen-binding fragment thereof, and (iii) an antibody against TF or an antigen-binding fragment thereof. Further, in a particular embodiment, said antibody is a monoclonal antibody.

Therefore, for its administration to a subject, said EPR-1 effector, such as an EPR-1 inhibitor or antagonist, hereinafter referred to as the “active ingredient”, will be formulated in a suitable pharmaceutical administration form, to which end the pharmaceutically acceptable carriers and excipients suitable for the preparation of the desired pharmaceutical administration form will be incorporated. Information about said carriers and excipients, as well as about said administration forms suitable for the administration of said product of the invention, can be found in galenic pharmacy treatises. A review of the different pharmaceutical administration forms of drugs in general, and of their preparation processes, can be found in the book “Tratado de Farmacia Galénica” (“Galenic Pharmacy Treatise”), by C. Faulí i Trillo, 1^{st} Edition, 1993, Luzán 5, S.A. of Ediciones.

The term “pharmaceutically acceptable carrier”, as used herein, is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In an embodiment, when the active ingredient is an antibody, it will be prepared with carriers that will protect said compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art.

The administration route of the active ingredient may be oral (e.g., solid or liquid), parenteral (including intravenous, intraperitoneal, intramuscular, subcutaneous, rectal or vaginal administration), by inhalation or topical.

The active ingredient will be present in the pharmaceutical composition in a therapeutically effective amount. Said amount may vary within a wide range depending of the nature and activity of the active ingredient. Generally an effective administered amount of the active ingredient will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.001 to 1,000 mg/kg body weight/day, preferably about 0.01 to about 100 mg/kg body weight/day, most preferably from about 0.05 to 10 mg/kg body weight/day. Since the the active ingredient dose to be administered to a subject will depend on a number of factors, the doses mentioned herein must be considered only as guides for a person skilled in the art, and this person must adjust the doses according to the previously mentioned variables. Nevertheless, the pharmaceutical composition of the invention can be administered one or more times a day for preventive or therapeutic purposes.

The pharmaceutical composition provided by this invention can be used together with other additional drugs useful in the prevention and/or treatment of a condition associated with platelet activation and/or platelet aggregation, to provide a combination therapy. Said additional drugs can be part of the same pharmaceutical composition or, alternatively, they can be provided in the form of a separate composition for their simultaneous or successive (sequential in time) administration with respect to the administration of the pharmaceutical composition provided by the invention.

The pharmaceutical composition provided by the invention can be used as an antiplatelet composition; as such, it can be used, for example, for the prevention and treatment of conditions associated with platelet activation and/or platelet aggregation, as well as in the prevention and/or treatment of thrombus or embolus mediated diseases.

Thus, in other aspect, the invention relates to a method for treating a condition associated with platelet activation and/or platelet aggregation, or a thrombus or embolus mediated disease, which comprises administering to a subject in need of said treatment a therapeutically effective amount of an EPR-1 effector, such as an EPR-1 inhibitor or antagonist, for example an antibody or an antigen-binding fragment thereof, that exhibits a specific binding activity for EPR-1, or for an EPR-1 ligand (e.g., FXa) or for a stimulator of an EPR-1 ligand (e.g., TF). Thus, in a particular embodiment, said antibody is selected from the group consisting of (i) an antibody against EPR-1 or an antigen-binding fragment thereof, (ii) an antibody against FXa or an antigen-binding fragment thereof, and (iii) an antibody against TF or an antigen-binding fragment thereof. Further, in a particular embodiment, said antibody is a monoclonal antibody. Illustrative, non-limitative, conditions or diseases to be treated according to the invention include, among others, conditions or disorders associated with an inappropriate platelet activation, e.g., atherosclerosis, coronary vascular disease, cerebrovascular disease, etc.; conditions or disorders associated with an abnormal platelet activation, e.g., atrial fibrillation, cancer, peripheral vascular disease, Alzheimer disease, inflammatory bowel disorders and recently with deep vein thrombosis, etc.; a thromboembolic disease, e.g, coronary heart disease, acute deep venous thrombosis, pulmonary embolism, acute arterial embolization of the extremities, myocardial infarction, disseminated intravascular coagulation, coronary artery disease, peripheral and cerebrovascular disease, intermittent claudication, thromboembolic complications of surgery (during as well as after invasive coronary interventions), or recurrence of embolism in patients with rheumatic and arteriosclerotic heart disease.

As mentioned above, said EPR-1 inhibitor or antagonist can be used to form a non-thrombogenic coating in the surface of a medical device used during said surgical operation. Thus, in other aspect, the invention relates to a method for forming a non-thrombogenic coating in the surface of a medical device for surgical operations which comprises contacting the surface of said medical device with an EPR-1 inhibitor or antagonist. Virtually, any medical device for surgical operations can be coated an EPR-1 inhibitor or antagonist in order to form a non-thrombogenic coating in the surface of said medical device; nevertheless, in a particular embodiment, said medical device is a medical device for use in surgery, for example, in hip replacement, angioplasty (e.g., coronary, pulmonary, peripheral, intracranial, extracranial carotid, renal, or aortic angioplasty), invasive cardiovascular surgery, e.g., coronary artery bypass graft or heart valve replacement, among others.

In an embodiment said EPR-1 inhibitor or antagonist is an antibody or an antigen-binding fragment thereof, that exhibits a specific binding activity for EPR-1, or for an EPR-1 ligand (e.g., FXa) or for a stimulator of an EPR-1 ligand (e.g., TF). Thus, in a particular embodiment, said antibody is selected from the group consisting of (i) an antibody against EPR-1 or an antigen-binding fragment thereof, (ii) an antibody against FXa or an antigen-binding fragment thereof, and (iii) an antibody against TF or an antigen-binding fragment thereof. Further, in a particular embodiment, said antibody is a monoclonal antibody.

### EXAMPLE

### Role of EPR-1 on the FXa-Induced Platelet Aggregation

The effect of FXa on platelet function was evaluated in washed platelet suspensions by the following assays:
Assays demonstrating that FXa directly causes platelet aggregation without thrombin formation.
Assays demonstrating synergistic effects between FXa and thrombin.
Blocking assays using antibodies against FXa and its receptor EPR-1.

### TF as a Stimulator of FXa Proteolytic Activity

The effect of lipidated TF on FXa proteolytic activity (amidolytic and thrombin formation activities) was evaluated in washed platelet suspensions by the following assays:
Assays demonstrating that in the absence of FVII, TF acts as a stimulator of FXa amidolytic (chromogenic assays using S-2765).
Assays demonstrating that in the absence of FVII, TF acts as a stimulator of FXa thrombin formation activity (chromogenic assays using S-2238).

### TF as a Stimulator of FXa-Induced Platelet Aggregation

The effect of lipidated TF on FXa platelet aggregatory activity was evaluated in washed platelet suspensions by the following assays:
Assays demonstrating that TF causes platelet aggregation in a washed activated platelet suspension (expressing p-selectin).
Assays demonstrating that TF acts as a stimulator of FXa platelet aggregatory activity and this effect is mediated by FXa-EPR-1 interaction.

### I. Materials and Methods

Materials

Commercial polyclonal antibody against EPR-1 was purchased from Calbiochem® (Anti-Effector Cell protease Receptor-1, Human (Rabbit)).

Commercial monoclonal antibody against FXa was purchased from SigmaBioSciences (Monoclonal Anti-Human Factor X Purified Mouse Immunoglobulin Clone HX-1).

Commercial monoclonal antibody against TF was purchased from BD Biosciences (BD Pharmigen™, Purified Mouse Anti-Human Monoclonal Antibody).

As a source of lipidated TF, Human Recombinant Tissue Factor (Non Lipidated) (American Diagnostica, USA) was used, relipidated following the method described by Morrissey.

Haematologic Technologies commercial compounds were used as sources of FXa and FII.

Commercial FX Coagulation Factor deficient plasma was purchased from Dade Behring Marburg GmbH.

Methods

Method for Relipidating TF in Lipid Vesicles Using Dialysis with Octylglucoside (Morrisey Method)

Non lipidated TF is incorporated into lipid vesicles using non ionic detergent N-octyl-beta-D-glucopyranoside (octylglucoside). Both TF and the lipids are dissolved in octylglucoside forming micelles. Octylglucoside can be easily removed from the solution by dialysis due to its high critical micelle concentration (CMC=20 to 50 mM). When the octylglucoside is removed the lipids are organized in unilamellar vesicles. TF is soaked in these vesicles by virtue of its transmembrane domain. Normally, 50 to 80% of TF molecules are arranged facing outwards from the vesicles.

Buffers and Stock Solutions

Octylglucoside (n-octyl-beta-D-glucopyranoside) from Calbiochem.

Lipids:

| | | | | Molecular |
|---|---|---|---|---|
| | Lipid | Concentration | | weight |
| PC | L-alpha-phosphatidylcholine | 10 or 25 | mg/ml | 761 |
| PS | L-alpha-phosphatidylserine, | 10 | mg/ml | 810 |
| | bovine brain sodium salt | | | |
| PE | L-alpha- | 10 | mg/ml | 768 |
| | phosphatidylethanolamine, | | | |
| | bovine liver | | | |

Buffers:

HBS
100 mM NaCl
20 mM Hepes/NaOH, pH 7.5
0.02% (m/v) sodium azide
HBSA (keep at 4° C.)
Bovine serum albumin in 0.1% (m/v) HBS
OG/HBS (prepare at time of use)
n-octyl-beta-D-glucopyranoside in 100 mM HBS (29.2 mg OG/ml
of HBS)

Preparing the Lipid Solution in Octylglucoside (OG)
1. 2.6 micromols of total lipids are prepared for each sample in a glass tube, using the desired lipid molar radius.
2. Dry the lipid mixture under an argon or nitrogen current.
3. When the tube appears to be dry, vacuum dry another 60 minutes.

4. Add 400 μl of a fresh solution of OG/HBS (at room temperature) to the tube containing the dry lipids.

For PC:PS vesicles (80:20 molar ratio)

| | | |
|---|---|---|
| 62 μl PC (at 25 mg/ml) | = 1.58 mg | = 2.08 μmol |
| 42 μl PS (at 10 mg/ml) | = 0.42 mg | = 0.52 μmol |

For PC:PE:PC vesicles (40:40:20 molar ratio)

| | | |
|---|---|---|
| 32 μl PC (at 25 mg/ml) | = 0.79 mg | = 1.04 μmol |
| 80 μl PE (at 10 mg/ml) | = 0.80 mg | = 1.04 μmol |
| 42 μl PS (at 10 mg/ml) | = 0.42 mg | = 0.52 μmol |

Relipidating

Add the desired amount of TF to the tube containing the 400 μl of OG/lipids and enough HBSA up to completing the final volume of 1 ml. Carry out this step at room temperature.

Obtaining Washed Platelet Suspensions

Washed platelet suspensions were prepared according to the method described by Radomski M. et al. (Radomski M, Moncada S.; 1983 Thromb Res. An improved method for washing of human platelets with prostacyclin. 15;30(4):383-9) from blood extractions (3.15% sodium citrate) from healthy volunteers. Processing of the specimens was always carried out immediately after blood extraction and at room temperature. Platelet activation and functionality states were assayed by means of aggregation assays prior to and during the performance of the assays. Self-activation and functionality were estimated by means of activation with a known agonist (collagen).

Platelet Aggregation Assays

Platelet aggregation assays were performed in washed platelets to prevent interference from plasma coagulation factors, obtained following the previously described protocol. These assays were performed by means of an optical method, following the aggregometer handling protocol (Chromogenix). All the information was processed by the Aggro/link interface which was recorded on a Pentium III computer. In short, 500 μl of washed platelets were added in the cuvettes along with the stirring magnet and they were tempered for 1 minute at 37° C. They were arranged in the aggregometer along with a cuvette for the blank (platelet suspension buffer), the reagents including antibodies to be analyzed were added in two of the channels and a reference reagent in the third, and aggregation time was recorded. The effect of monoclonal antibody against TF was evaluated after 1 hour incubation at room temperature.

In Vitro Assays

Chromogenic Assays

Different chromogenic assays were designed in washed platelet suspensions to demonstrate the effect of rTF on factor Xa proteolytic activity.

FXa amidolytic activity was determined by means of a chromogenic assay using S-2765 (Chromogenix) as the chromogenic substrate for FXa, whereas thrombin forming activity was analyzed using S-2238 (Chromogenix) as the chromogenic substrate for thrombin.

The chromogenic assays were performed in washed platelet suspensions. Suitable volumes of each one of the factors to be studied were dispensed on an ELISA plate and a suitable volume such as to have a concentration of 250,000 platelets/μl in the medium. Finally, adding the specific chromogenic substrate allowed quantifying the proteolytic activity in question by means of spectrophotometric readings at 405 nm. In the assays for determining amidolytic activity only FXa and the different rTF concentrations were dispensed, whereas in the assays for determining thrombin forming activity it was necessary to further add factor II (prothrombin) and without FVa (given that in the washed platelet assays these already contained endogenous FVa).

Coagulation Assays in Plasma

Spontaneous procoagulant activity (unstimulated) in plasma was measured by means of a coagulation assay with a certain step in a coagulometer (Fibrintimer BFT-II clot-timer Dade-Behring, Germany). In short, 50 μl of platelet-poor plasma were added to the already tempered cuvettes and 50 μl of distilled water were added. This mixture was left to incubate for 60 seconds at 37° C. and 50 μl of 25 mM calcium chloride were immediately added and the coagulation time was determined in seconds in the coagulometer, verified by formation of the clot. Each one of the samples was assayed in duplicate.

### II. Results

### A. Role of EPR-1 on the FXa-Induced Platelet Aggregation

Several assays were performed for the purpose of evaluating the role of EPR-1 on the FXa-induced platelet aggregation.

### A.1. Assays Demonstrating that FXa Directly Causes Platelet Aggregation without Thrombin Formation

FXa-Induced Platelet Aggregation

The proaggregatory effect mediated by FXa was determined in washed platelet suspensions. Table 1 shows the results obtained in three independent experiments. Platelet aggregation was measured in an aggregometer at 12 minutes after the addition of reagents and the percentage of aggregation was calculated. FXa was able to induce a strong proaggregatory effect at very low concentration (below 10 pM).

TABLE 1
FXa induces platelet aggregation in washed platelet suspension

| FXa | Platelet aggregation (%) |
|---|---|
| 100 pM | 100 |
| 20 pM | 100 |
| 10 pM | 100 |
| 2 pM | 67.5 |
| 1 pM | 30 |
| 0.2 pM | 0 |

Mean ± SEM (n = 3)

Chromogenic Assays for Thrombin Forming Activity in Washed Platelet Suspensions

The procoagulant effect detected of FXa by thrombin formation was determined by means of specific substrate S-2238 (table 2). In the absence of exogenous FXa, no thrombin formation was detected. No effects were observed when FXa was present at low concentrations (12.5 pM). A very slightly effect was observed at concentrations between 125 pM and 250 pM. And more significant effects were observed in the presence of exogenous FXa at higher concentrations (1,250 pM).

TABLE 2
FXa induces thrombin formation in washed platelets

| | |
|---|---|
| FXa 1,250 pM | 421 ± 19 |
| FXa 250 pM | 213 ± 21 |
| FXa 125 pM | 201 ± 29 |
| FXa 12.5 pM | 0 |
| FXa absent | 0 |

Mean ± SEM (n = 5)

Plasmatic Coagulation Assays in Plasma of FXa Deficient Plasma for Titulation of FXa.

A series of in vitro coagulation assays were performed showing the coagulating effect of FXa in FXa depleted plasma at different concentrations (table 3). Similarly to thrombin forming activity studies in washed platelet suspensions, concentrations below 1.7 nM of FXa, no significant procoagulant effects were observed by addition of exogenous FXa.

TABLE 3

Effect of FXa in plasmas depleted of FXa

| | |
|---|---|
| FXa 1.7 μM | 25 |
| FXa 170 nM | 42 |
| FXa 17 nM | 75.9 |
| FXa 1.7 nM | 133.6 |
| FXa 170 pM | 280.2 |
| FXa 17 pM | >400 (non coagulated) |
| Without FXa | >400 (non coagulated) |

Mean ± SEM (n = 5)

### A.2. Assays Demonstrating Synergistic Proaggregatory Effects Between FXa and Thrombin

A curve dose response of thrombin proaggregatory effect is shown in table 4. Results showing the synergistic effect between both serine proteases are also shown. Platelet aggregation was observed at very low concentrations (thrombin at 10 mU/ml and FXa at 0.01 mU/ml, both unable to cause platelet aggregation by themselves. Platelet aggregation was measured in a aggregometer at 12 minutes after the addition of reagents and the percentage of aggregation was calculated.

TABLE 4
Synergistic effect between FXa and thrombin

| | | Platelet aggregation (%) |
|---|---|---|
| Thrombin (mU/ml) | 40 | 100 |
| | 30 | 100 |
| | 20 | 50 |
| | 10 | 0 |
| | 5 | 0 |
| FXa (mU/ml) | 0.01 | 0 |
| (corresponding to 0.2 pM) | | |
| Thrombin 10 mU/ml + FXa | | 81 |
| 0.01 mU/ml | | |

Mean ± SEM (n = 3)

### A.3. Blocking Assays Using Antibodies Against FXa and its Receptor EPR-1

The specificity of FXa-induced platelet aggregation effect was demonstrated using monoclonal antibodies against FXa (table 5). In the presence of 24 μg/ml of monoclonal anti-human Factor Xa Purified Mouse Immunoglobulin Clone HX-1, the proaggregatory effect mediated by FXa was fully blocked. On the other hand, polyclonal antibodies against EPR-1 were able to fully block the FXa-induced platelet aggregation.

TABLE 5
FXa induces platelet aggregation in washed platelet suspension

| | | Anti FXa | |
|---|---|---|---|
| FXa | Platelet aggregation (%) | 24 μg/ml | Anti EPR-1 20 μg/ml |
| 100 pM | 100 | | |
| 20 pM | 100 | | |
| 15 pM | 100 | 0 | 0 |
| 10 pM | 100 | | |
| 2 pM | 67.5 | | |
| 1 pM | 30 | | |
| 0.2 pM | 0 | | |

Mean ± SEM (n = 3)

### B. Lipidated TF as a Stimulator of FXa Proteolytic Activity

Several in vitro assays were performed for the purpose of evaluating lipidated TF capacity as a factor Xa stimulating agent in the absence of FVII: (i) chromogenic assays for FXa amidolytic activity in solution; and (ii) chromogenic assays for thrombin forming activity in washed platelet suspension.

### B.1. Assays Demonstrating that in the Absence of FVII, TF Acts as a Stimulator of FXa Amidolytic

FXa Amidolytic Activity in Washed Platelet Suspension

Chromogenic studies using S-2765 as specific substrate for FXa are shown in table 6. Results obtained from five independent experiments demonstrate that in the presence of very low exogenous FXa concentrations, rTF caused a significant stimulating effect on the amidolytic activity evaluated with specific substrate S-2765 (p<0.001). Similar stimulating effects were observed in the presence of high FXa concentrations.

TABLE 6
TF induces FXa amidolytic activity (S-2765) expressed in washed platelets

| | Without | | | |
|---|---|---|---|---|
| | rTF | rTF 10 μg/ml | rTF 1 μg/ml | rTF 0.1 μg/ml |
| FXa 6,800 pM | 220 ± 12 | 706 ± 32 | 655 ± 41 | 291 ± 33 |
| FXa 1,400 pM | 26 ± 3 | 289 ± 11 | 311 ± 22 | 165 ± 28 |
| FXa 700 pM | 9 ± 2 | 341 ± 14 | 306 ± 19 | 143 ± 14 |

Mean ± SEM (n = 5)

### B.2. Assays Demonstrating that in the Absence of FVII, TF Acts as a Stimulator of FXa Thrombin Formation Activity (Chromogenic Assays Using S-2238)

FXa Thrombin Forming Activity in Washed Platelet Suspensions

Chromogenic studies using S-2238 as specific substrate for thrombin are shown in table 7. In the absence of exogenous FXa, lipidated TF produced a significant stimulating effect on thrombin formation (p<0.001). These results indicate that intraplatelet FXa was expressed in platelet surface and thrombin formation took place. Similar stimulating effects were observed in the presence of exogenous FXa both at low and high concentrations (p<0.001).

TABLE 7
Lipidated TF induces thrombin formation in washed platelets in the
presence of FII but in the absence of FVa (already present in platelets) and
FVII

| | Without rTF | rTF 0.1 μg/ml | rTF 1 μg/ml |
|---|---|---|---|
| FXa 1,250 pM | 421 ± 19 | 13,854 ± 145 | 14,910 ± 168 |
| FXa 250 pM | 213 ± 21 | 9,324 ± 155 | 9,610 ± 114 |
| FXa 125 pM | 201 ± 29 | 7,802 ± 113 | 8,508 ± 178 |
| FXa absent | 0 | 906 ± 89 | 3,504 ± 69 |

Mean ± SEM (n = 5)

### C. TF as a Stimulator of FXa-Induced Platelet Aggregation

### C.1. Assays Demonstrating that TF Causes Platelet Aggregation in a Washed Activated Platelet Suspension (Expressing p-Selectin)

The proaggregatory effect mediated by TF was determined in washed platelet suspensions. Table 8 shows the results obtained in three independent experiments. Platelet aggregation was measured in a aggregometer at 16 minutes after the addition of reagents and the percentage of aggregation was calculated. TF was able to induce a strong proaggregatory effect at very low concentration (10 ng/ml) in suspensions containing washed activated platelets (monitored by flow cytometry using monoclonal antibodies against p-selectin).

TABLE 8
Effect of FXa in plasmas depleted of FXa

| rTF (ng/ml) | Platelet aggregation (%) |
|---|---|
| 250 | 100 |
| 50 | 100 |
| 10 | 83 |
| 5 | 78 |
| 1 | 0 |

Mean ± SEM (n = 3)

### C.2. Assays Demonstrating that TF Acts as a Stimulator of FXa Platelet Aggregatory Activity and this Effect is Mediated by FXa-EPR-1 Interaction

Table 9 lists the assay results in which TF in the absence of exogenous FXa is able to induce platelet aggregation and that this is mediated by FXa through signaling of its receptor, membrane receptor EPR-1. The inhibiting effect achieved with antibodies directed against FXa, TF and receptor EPR-1 demonstrates that TF interacts with FXa exposed on the platelet surface, that FXa interacts with FXa-specific receptor EPR-1, and that all of them (EPR-1, FXa, TF) are essential for the early non-trombin dependent platelet aggregation which mediates platelet aggregation by means of the action of this proteolytic enzyme, and that said aggregation is signaled by the FXa-specific receptor, EPR-1. Accordingly, formation of the TF:FXa complex on the platelet surface, as well as producing thrombin formation, produces platelet aggregation and further enhances hemostasic clot formation. For the assay with anti-TF, the antibody was incubated with the source of TF for 1 h at room temperature prior to its addition as a reagent.

TABLE 9
rTF induces platelet aggregation mediated by FXa through
the signaling of its receptor (EPR-1)

| | rTF concentration [ng/ml] | | | | |
|---|---|---|---|---|---|
| | Without rTF | 5 | 10 | 100 | 250 |
| Without FXa | 0 | 80 | 100 | 100 | 100 |
| FXa 8 pg/ml | 0 | 100 | 100 | 100 | 100 |
| FXa 80 pg/ml | 0 | 100 | 100 | 100 | 100 |
| FXa 240 pg/ml | 100 | 100 | 100 | 100 | 100 |
| anti FXa (24 μg/ml) + FXa | 0 | | | 0 | |
| 240 pg/ml | | | | | |
| anti EPR-1 (20 μg/ml) + FXa | 0 | | | 0 | |
| 240 pg/ml | | | | | |
| anti TF (50 μg/ml) + FXa | 0 | | | 0 | |
| 240 pg/ml | | | | | |

Mean ± SEM (n = 5)

1. A method of regulating platelet activation and aggregation in a subject, said method comprising administering an effective amount of an antiplatelet pharmaceutical composition to a subject, said antiplatelet pharmaceutical composition comprising an effector cell protease receptor-1 (EPR-1) effector.

2. The method according to claim 1, wherein said antiplatelet pharmaceutical composition is for the prevention and/or treatment of a condition associated with platelet aggregation in a subject.

3. The method according to claim 1, wherein said EPR-1 effector is selected from the group consisting of a direct EPR-1 effector and an indirect EPR-1 indirect effector.

4. The method according to claim 3, wherein said indirect EPR-1 effector is selected from the group consisting of an effector of an EPR-1 ligand and an effector of a stimulator of an EPR-1 ligand.

5. The method according to claim 4, wherein said indirect EPR-1 effector is selected from the group consisting of an activated factor X (FXa) effector and a tisular factor (TF) effector.

6. The method according to claim 3, wherein said EPR-1 effector is an EPR-1 inhibitor or an EPR-1 antagonist.

7. The method according to claim 6, wherein said EPR-1 inhibitor or antagonist is present in the antiplatelet pharmaceutical composition for inhibiting a condition in the subject selected from the group consisting of platelet activation, platelet aggregation, and thrombus formation.

8. The method according to claim 6, wherein said EPR-1 inhibitor or antagonist is present in the antiplatelet pharmaceutical composition for preventing and/or treating a condition in the subject selected from the group consisting of a cardiovascular disease related to thrombosis, a cardiovascular disease related to embolism, a platelet associated thromboembolic disease that is primarily arterial in origin, a recurring myocardial infarction and stroke, a recurring embolism in patients with rheumatic and arteriosclerotic heart disease, acute deep venous thrombosis, pulmonary embolism, acute arterial embolization of the extremities, myocardial infarction, disseminated intravascular coagulation, coronary artery disease, cerebrovascular disease, cancer, Alzheimer disease, and atrial fibrillation.

9. The method according to claim 6, wherein said EPR-1 inhibitor or antagonist is present in the antiplatelet pharmaceutical composition for the prevention and/or treatment of thromboembolic complications of surgery.

10. The method according to claim 9, wherein said surgery includes hip replacement, angioplasty or invasive cardiovascular surgery.

11. The method according to claim 10, wherein said invasive cardiovascular surgery includes coronary artery bypass graft or heart valve replacement.

12. The method according to claim 10, wherein said angioplasty is selected from the group consisting of coronary, pulmonary, peripheral, intracranial, extracranial carotid, renal, and aortic angioplasty.

13. The method according to claim 8, wherein said EPR-1 inhibitor or antagonist for the treatment of cancer is used in combination with at least one antineoplasic agent and a pharmaceutically acceptable carrier.

14. The method according to claim 8, wherein said EPR-1 inhibitor or antagonist for the treatment of Alzheimer disease is used in combination with at least one additional drug and a pharmaceutically acceptable carrier.

15. The method according to claim 8, wherein said EPR-1 inhibitor or antagonist for the treatment of atrial fibrillation is used in combination with at least one additional drug and a pharmaceutically acceptable carrier.

16. The method according to claim 6, wherein said EPR-1 inhibitor or antagonist is an antibody or an antigen-binding fragment thereof, that exhibits a specific binding activity for EPR-1, for an EPR-1 ligand, or for a stimulator of an EPR-1 ligand.

17. The method according to claim 16, wherein said antibody is selected from the group consisting of: an antibody against EPR-1; an antigen-binding fragment of an antibody against EPR-1; an antibody against FXa; an antigen-binding fragment of an antibody against FXa; an antibody against TF; and an antigen-binding fragment of an antibody against TF.

18. The method according to claim 17, wherein said antibody is a monoclonal antibody.

19. A method for forming a non-thrombogenic coating in the surface of a medical device for surgical operations which comprises contacting the surface of said medical device with an effector cell protease receptor-1 (EPR-1) inhibitor or antagonist.

20. The method according to claim 19, wherein said EPR-1 inhibitor or antagonist is an antibody or an antigen-binding fragment thereof, that exhibits a specific binding activity for EPR-1, for an EPR-1 ligand, or for a stimulator of an EPR-1 ligand.

21. The method according to claim 20, wherein said antibody is selected from the group consisting of: an antibody against EPR-1; an antigen-binding fragment of an antibody against EPR-1; an antibody against FXa; an antigen-binding fragment of an antibody against FXa; an antibody against TF; and an antigen-binding fragment of an antibody against TF.

22. The method according to claim 21, wherein said antibody is a monoclonal antibody.

23. A method for treating a condition associated with platelet activation and/or platelet aggregation, or a thrombus or embolus mediated disease, which comprises administering to a subject in need of said treatment a therapeutically effective amount of an effector cell protease receptor-1 (EPR-1) inhibitor or antagonist.

24. The method according to claim 23, wherein said EPR-1 inhibitor or antagonist is an antibody or an antigen-binding fragment thereof, that exhibits a specific binding activity for EPR-1, for an EPR-1 ligand, or for a stimulator of an EPR-1 ligand.

25. The method according to claim 24, wherein said antibody is selected from the group consisting of: an antibody against EPR-1; an antigen-binding fragment of an antibody against EPR-1; an antibody against FXa; an antigen-binding fragment of an antibody against FXa; an antibody against TF; and an antigen-binding fragment of an antibody against TF.

26. The method according to 25, wherein said antibody is a monoclonal antibody.

27. The method according to claim 23, wherein said condition to be treated is selected from the group consisting of atherosclerosis, coronary vascular disease, cerebrovascular disease, atrial fibrillation, cancer, peripheral vascular disease, Alzheimer disease, inflammatory bowel disorders, deep vein thrombosis, coronary heart disease, acute deep venous thrombosis, pulmonary embolism, acute arterial embolization of the extremities, myocardial infarction, disseminated intravascular coagulation, coronary artery disease, peripheral and cerebrovascular disease, intermittent claudication, thromboembolic complications of surgery and the recurrence of embolism in patients with rheumatic and arteriosclerotic heart disease.
